(19) United States
(12) Patent Application Publication
(10) Pub. No.: US 2020/0297684 A1
(43) Pub. Date: Sep. 24, 2020

(54) TOPICAL COMPOSITIONS AND METHODS FOR TREATING ACNE VULGARIS

(71) Applicant: Bausch Health Ireland Limited, Dublin (IE)

(72) Inventors: Radhakrishnan Pillai, Santa Rosa, CA (US); Varsha D. Bhatt, San Francisco, CA (US)

(21) Appl. No.: 16/822,831

(22) Filed: Mar. 18, 2020

Related U.S. Application Data
(60) Provisional application No. 62/819,880, filed on Mar. 18, 2019.

(51) Int. Cl. A61K 31/203; A61K 31/327; A61P 17/10

(52) U.S. Cl. CPC A61K 31/203; A61K 9/0014; A61P 17/10; A61K 31/327

(57) ABSTRACT

Topical pharmaceutical compositions comprise a combination of retinoid and an antibacterial agent. In particular, a topical composition comprises tretinoin and benzoyl peroxide. Such compositions are used for treating acne vulgaris.

### BACKGROUND OF THE INVENTION

The present invention relates to topical compositions and methods for treating acne vulgaris. In particular, this invention relates to topical pharmaceutical compositions comprising a combination of active ingredients, and methods using the same, for treating acne vulgaris. More particularly, this invention relates to topical pharmaceutical compositions comprising a combination of an anti-bacterial agent and a retinoid, and methods using the same, for treating acne vulgaris.

Acne vulgaris (or commonly referred to as acne) is a skin disease affecting more than ninety percent of the population world-wide. Up to fifty million people between the ages of 12 and 24 in the United States experience at least minor acne annually. Acne vulgaris is characterized by skin with scaly red skin (seborrhea), blackheads and whiteheads (open and closed comedones, respectively), pinheads (papules), large papules (nodules), and pustules, which can lead to scarring. The pathogenesis of acne is multifactored, involving seborrhea, microbial proliferation, inflammation, and abnormal desquamation of follicular epithelium. Excessive sebum production, brought about by hormonal changes (in particular, an increase in the production of androgens associated with the onset of puberty) is followed by abnormal desquamation of follicular corneocytes. The mixture of cells and sebum creates an environment for the proliferation of Propionibacterium acnes. Chemotactic factors released by P. acnes attract lymphocytes and neutrophils, as well as producing other pro-inflammatory molecules that lead to inflammation.

Acne is not life threatening but severe acne can affect psychological status and social activities.

Common current treatments for acne include oral antibiotics for inflammatory acne, oral retinoids for severe acne, topical antibiotics and topical medications with bacteriostatic, anti-inflammatory, or keratolytic properties for mild-to-moderate inflammatory acne.

These treatments have been known to produce side effects. For example, side effects of oral antibiotics include nausea, vomiting, diarrhea, abdominal pain and cramps, pruritus, rash, stomatitis, and dizziness. Common side effects of oral retinoids include cheilitis, dry skin and mucous membranes, pruritus, epistasis, and photosensitivity. Side effects of topical antibiotics include local irritation. Side effects of topical retinoids include dryness, scaling, erythema, burning, irritation, and photosensitivity. See; e.g., S. Feldman et al., Am. Fam. Physician (2004), 69(9): 2123-30. The severity of side effects can vary with the retinoids, formulations, and dosages.

The need still exists for more effective and safer topical medicaments with reduced adverse effects for the management of acne vulgaris.

### SUMMARY OF THE INVENTION

In general, the present invention provides topical compositions and methods for treating acne vulgaris.

In one aspect, the present invention provides topical pharmaceutical compositions comprising a combination of an anti-bacterial agent and a retinoid, and methods using the same, for treating acne vulgaris.

In another aspect, the present invention provides topical pharmaceutical compositions comprising a combination of: (a) tretinoin or a pharmaceutically acceptable salt or ester thereof; and (b) benzoyl peroxide. The present invention also provides methods using the compositions, for treating acne vulgaris.

In still another aspect, the present invention relates to topical pharmaceutical compositions comprising a combination that is an admixture of a first composition (“Component 1”) comprising tretinoin or a pharmaceutically acceptable salt or ester thereof, and a second composition (“Component 2”) comprising benzoyl peroxide. In yet another aspect, the admixture is formed or prepared substantially immediately prior to the admixture being applied to a subject in need of treatment.

In still another aspect of the present invention, the admixture comprises, or is formed or prepared from, substantially equal amounts of the first composition (Component 1) and the second composition (Component 2) substantially immediately prior to the admixture being applied to a subject in need of treatment.

In yet another aspect, the admixture comprises tretinoin at about 0.01-0.1 weight percent of the total admixture and benzoyl peroxide at about 1-10 weight percent of the total admixture.

In still another aspect of the present invention, the first composition (Component 1) and the second composition (Component 2) comprise aqueous gels.

In one embodiment, the aqueous gels comprise a gelling agent that comprises a carboxyvinyl polymer, such as poly(acrylic acid) or a derivative thereof. In another embodiment, such gelling agent comprises a cross-linked poly(acrylic acid).

In still another aspect, the present invention provides a method of treating or ameliorating acne vulgaris in a subject. The method comprises topically administering to an affected area of the subject's body a composition comprising a combination of (a) and tretinoin at about 0.1-0.1 weight percent of the combination; and (b) benzoyl peroxide at about 1-10 weight percent of the combination; in an amount and for a time sufficient to treat or ameliorate such acne vulgaris.

Other features and advantages of the present invention will become apparent from the following detailed description and claims.

### DETAILED DESCRIPTION OF THE INVENTION

In general, the present invention provides topical compositions and methods for treating acne vulgaris.

Throughout this disclosure, unless otherwise indicated, the concentration of an ingredient of a composition indicated as a percentage is in percent by weight of the total composition.

In one aspect, the present invention provides topical pharmaceutical compositions comprising a combination of an anti-bacterial agent and a retinoid, and methods using the same, for treating acne vulgaris. Non-limiting examples of retinoids are tretinoin, isotretinoin, tazarotene, bexarotene, adapalene, etretinate and acitretin. The preferred retinoid is tretinoin.

In another aspect, the present invention provides topical pharmaceutical compositions comprising a combination of: (a) tretinoin or a pharmaceutically acceptable salt or ester thereof; and (b) benzoyl peroxide. The present invention also provides methods using such compositions, for treating acne vulgaris.

In still another aspect, the present invention relates to topical pharmaceutical compositions comprising a combination that is an admixture of a first composition (Component 1) comprising tretinoin or a pharmaceutically acceptable salt or ester thereof, and a second composition (Component 2) comprising benzoyl peroxide. In yet another aspect, the admixture is formed or prepared substantially immediately prior to the admixture being applied to a subject in need of treatment. In other words, an amount of Component 1 and another amount of component 2 are mixed together to form the admixture substantially immediately prior to applying the admixture to the subject.

Since it has been known that tretinoin can be inactivated by ultraviolet (UV) light and is susceptible to oxidization by a strong oxidizing agent such as benzoyl peroxide, the prior art has recommended that, if they are to be used in the same regimen to treat acne vulgaris, they should be applied at different times. For example, tretinoin should be applied at night and never together with benzoyl peroxide. See; e.g., S. Feldman et al., Am. Fam. Physician (2004), 69(9):2123-30.

Although Del Rosso et al. (J. Clin. Aesthet. Dermatol. (2010), 3(10):26-28) found that tretinoin in a mixture of equal parts of a 0.05% tretinoin gel and a gel containing 5% BPO and clindamycin phosphate was stable at 32° C. for 7 hours, the stability of tretinoin beyond 7 hours or in a mixture without clindamycin phosphate was not known. The present inventors surprisingly discovered that treinoin in an admixture of tretinoin and benzoyl peroxide compositions of the present invention is chemically stable for at least 24 hours, even at 32° C. under room light condition, such that tretinoin and benzoyl peroxide can be administered together in the same composition to treat acne vulgaris. Such stability discovered by the present inventors allows for once-a-day treatment using tretinoin and benzoyl peroxide together in the same composition that has been taught away by Feldman et al. The method of the present invention provides synergistically enhanced efficacy in the treatment of acne vulgaris over the use of each of tretinoin and benzoyl peroxide alone. In other words, the present inventors surprisingly discovered that tretinoin and benzoyl peroxide could be applied together, such as in an admixture of the present invention, not only without evidence of loss of efficacy of either active ingredient, but moreover with enhanced efficacy.

The chemical stability of tretinoin in an admixture with benzoyl peroxide of the present invention at room temperature and 32 degrees C., under room light condition is shown in Table 1.

TABLE 1
Stability of Tretinoin at Room Condition
and at 32° C., Under Room Light

| Time | | Assay (% label claim) | |
|---|---|---|---|
| (hr) | Replicate | Room Condition | 32° C., Room Light |
| 0 | top | 98.8 | 98.8 |
| | middle | 97.1 | 97.1 |
| | bottom | 98.3 | 98.3 |
| 1 | 1 | 96.8 | 98.5 |
| | 2 | 97.2 | 97.1 |
| 3 | 1 | 97.8 | no data |
| | 2 | 97.5 | no data |
| 4 | 1 | no data | 98.6 |
| | 2 | no data | 98.2 |
| 5 | 1 | 97.0 | no data |
| | 2 | 97.3 | no data |
| 8 | 1 | 99.5 | no data |
| | 2 | 99.5 | no data |
| 12 | 1 | 97.3 | 98.9 |
| | 2 | 97.8 | 97.0 |
| 24 | 1 | 98.1 | 96.6 |
| | 2 | 97.4 | 97.8 |

More than 97 and 96 percent of treinoin in the original admixture of the present invention remains after 24 hours at room condition and 32° C., under room light, respectively. Such stability ensures that the therapeutic efficacy of tretinoin is not diminished between once-a-day applications.

In another aspect, a topical pharmaceutical admixture of the present invention is used to treat or ameliorate signs and symptoms of acne vulgaris in patients suffering from moderate to severe acne vulgaris.

In one aspect, the present invention relates to topical pharmaceutical compositions comprising a combination that is an admixture of a first composition (Component 1) comprising tretinoin or a pharmaceutically acceptable salt or ester thereof and a second composition (Component 2) comprising benzoyl peroxide. In yet another aspect, the admixture is formed or prepared substantially immediately prior to the admixture being applied to a subject in need of treatment.

In still another aspect of the present invention, the admixture comprises, or is formed or prepared from, Component A and Component B at a volume ratio of about 1:5 to about 5:1. In one embodiment the admixture comprises substantially equal volume amounts of Component 1 and Component 2. The admixture composition of the invention is prepared or formed substantially immediately prior to the admixture being applied to a subject in need of treatment. Regardless of what volume ratio of Component A and Component B is used, the desired amounts of active ingredients may be achieved, for example, in one embodiment wherein tretinoin is present in the admixture at a concentration of about 0.05 weight percent of the total admixture and benzoyl peroxide is present in the admixture at about 2.5 weight percent of the total admixture.

In one aspect, an amount of the admixture comprises amounts (by weight) of Component 1 and Component 2 such that, after mixing, tretinoin is present at a concentration of about 0.01-0.1 weight percent of the total admixture and benzoyl peroxide is present at about 1-10 weight percent of the total admixture. In some embodiments, after mixing, tretinoin is present at a concentration of about 0.03-0.08 (or 0.04-0.07, or 0.04-0.06) weight percent of the total admixture and benzoyl peroxide is present at about 1-10 (or 1.5-5, or 2-3) weight percent of the total admixture. In a preferred embodiment, after mixing, tretinoin is present at a concentration of about 0.05 weight percent of the total admixture and benzoyl peroxide is present at about 2.5 weight percent of the total admixture.

For example, separately, Component 1 comprises tretinoin at a concentration of 0.02-3 weight percent (or 0.02-2, or 0.02-1, or 0.02-0.5 weight percent), and Component 2 comprises benzoyl peroxide at a concentration of 2-20 weight percent (or 2-15, or 2-10, or 2-6 weight percent).

In one aspect, compositions of the present invention are in the dosage form of gel, emulsion (including lotion, cream, and milk), foam, suspension, liquid, spray, paste, or ointment. In certain preferred embodiments, compositions of the present invention comprise aqueous gels.

In addition to the active ingredients as disclosed above, compositions of the present invention comprise one or more dermatologically acceptable excipients, such as liquid oils, viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents, or cosolvents. The choice of excipients depends on the desired dosage form.

Compositions of the invention may desirably comprise a gelling agent to provide viscosity so that the compositions may be provided in the form of a gel. Preferably, but not necessarily, the gelling agent is miscible or soluble in an aqueous medium. Non-limiting examples of suitable gelling agents are carbomers (also known as carboxy vinyl polymers, which are cross-linked polyacrylic acid), such as Carbopol® and polycarbophil (The Lubrizol Corporation, Wickliffe, Ohio). Carbopol® homopolymers are polymers of acrylic acid crosslinked with allyl sucrose or allylpentaerythritol. Carbopol® copolymers are polymers of acrylic acid and C₁₀-C₃₀ alkyl acrylate crosslinked with allylpentaerythritol. Carbopol® interpolymers are carbomer homopolymers or copolymers that contain a block copolymer of polyethylene glycol and a long chain alkyl acid ester. Noveon® polycarbophil is a polymer of acrylic acid crosslinked with divinyl glycol.

In addition, compositions of the invention may desirably comprise one or more thickening agents. Non-limiting examples of such thickening agents include acacia, alginic acid and its salts, hyaluronic acid and its salts, carboxymethylcellulose, ethylcellulose, gelatin, collagen, hydroxyethyl cellulose, hydroxypropyl cellulose, hydroxypropylmethyl cellulose, methylcellulose, poloxamers, polyvinylpyrrolidone, polyvinyl alcohol, tragacanth, xanthan gum, magnesium aluminum silicate, and bentonite.

A surfactant or emulsifier is included, if desired or required. Pharmaceutically acceptable anionic, cationic, or non-ionic surfactants may be included in a composition of the present invention. Non-ionic surfactants are preferred. Non-limiting examples of non-ionic surfactants are Octoxynol (also known as Macrogol tetramethylbutylphenyl ether, octylphenoxy polyethoxyethanol, or polyoxyethylene octylphenyl ether), such as Octoxynol 1, 3, 5, 8, 9, 10, 12, 13, 16, 30, 40, 70 (wherein the number indicates the number of repeating oxyethylene units), or other Octoxynols that comprise different numbers of repeating units of oxyethylene in the side chain, sorbitan esters (such as sorbitan monooleate and sorbitan monostearate, commonly known by their trade names Span 80 and Span 60), polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108), or poloxamines (synthetic block polymers of ethylene oxide and propylene oxide attached to ethylene diamine, such as those commonly known by their trade names of Tetronic®; e.g., Tetronic® 1508 or Tetronic® 908, etc.), other nonionic surfactants such as Brij® (polyoxyethylene alkyl ether having a formula of CH₃—(CH₂)₁₀₋₁₆—(O—C₂H₄)₁₋₂₅—OH), Myrj® (stearic acid esterified with polyoxyethylene having 40-100 repeating oxyethylene units), and long chain fatty alcohols (e.g., oleyl alcohol, stearyl alcohol, myristyl alcohol, docosahexaenoyl alcohol, etc.) with carbon chains having about 12 or more carbon atoms (e.g., such as from about 12 to about 24 carbon atoms).

In addition, polymeric emulsifiers such as those known under the trade name Pemulen™ (The Lubrizol Corporation, Wickliffe, Ohio) may be used. These are polymers of acrylic acid, modified by long chain (C₁₀-C₃₀) alkyl acrylates, and crosslinked with allylpentaerythritol.

An anionic emulsifier may be used, such as sodium or potassium oleate, triethanolamine stearate, sodium lauryl sulfate, sodium dioctyl sulfosuccinate, and sodium docusate. Less preferred are cationic emulsifiers such as quaternary ammonium salts. Still other emulsifiers include glyceryl monostearate, polyoxyethylene monooleate, polyoxyethylene monostearate, polyoxyethylene monolaurate, potassium oleate, sodium lauryl sulfate, sodium oleate, sorbitan monolaurate, sorbitan monopalmitate, sorbitan monostearate, sorbitan tristearate, sorbitan monooleate, sorbitan trioleate, triethanolamine oleate, polyoxyethylene sorbitan monolaurate, polyoxyethylene sorbitan monopalmitate, polyoxyethylene sorbitan monostearate, polyoxyethylene sorbitan tristearate, polyoxyethylene sorbitan monooleate, and polyoxyethylene sorbitan trioleate.

Compositions of the present invention desirably contain a dermatologically acceptable humectant such as glycerin, sorbitol, hexylene glycol, propylene glycol, or urea. Chelating agents (such as EDTA and its salts), and antioxidants (such as butylated hydroxytoluene (BHT) butylated hydroxyanisole (BHA), sodium metabisulfite, propyl gallate, or cysteine) may be included in a composition of the present invention.

In one aspect, separately, Component 1 of the present invention comprises the ingredients at the concentrations shown in Table 2.

TABLE 2
Separate Component 1 of the Present Invention
for Treating Acne vulgaris

| | Concentration (weight percent) | | |
|---|---|---|---|
| Ingredient | Range 1 | Range 2 | Range 3 |
| Retinoid | 0.02-2 | 0.02-1 | 0.02-0.5 |
| Humectant | 5-20 | 5-15 | 7-12 |
| Gelling/Thickening Agent | 0.2-5 | 0.3-3 | 0.5-2 |
| Surfactant/Emulsifier | 0.02-2 | 0.02-1 | 0.05-0.5 |
| Moisturizer | 0.1-20 | 0.1-15 | 0.1-12 |
| Anti-oxidant | 0.01-1 | 0.01-0.7 | 0.01-0.5 |
| Neutralizing Agent | q.s. to adjust | q.s. to adjust | q.s. to adjust |
| | pH to 4-7 | pH to 4-7 | pH to 4-7 |
| Purified water | q.s. to 100 | q.s. to 100 | q.s. to 100 |

A non-limiting example of a Component 1 of the present invention comprises a gel comprising tretinoin at a concentration of 0.1 weight percent and a carboxyvinyl polymer gelling agent.

In one aspect, separately, Component 2 of the present invention comprises the ingredients at the concentrations shown in Table 3.

TABLE 3
Separate Component 2 of the Present Invention
for Treating Acne vulgaris

| | Concentration (weight percent) | | |
|---|---|---|---|
| Ingredient | Range 1 | Range 2 | Range 3 |
| Antibacterial Agent | 2-15 | 2-10 | 2-6 |
| Humectant | 5-20 | 5-15 | 5-12 |
| Gelling/Thickening Agent | 0.2-5 | 0.3-3 | 0.5-2 |
| Neutralizing Agent | q.s. to adjust | q.s. to adjust | q.s. to adjust |
| | pH to 4-7 | pH to 4-7 | pH to 4-7 |
| Purified water | q.s. to 100 | q.s. to 100 | q.s. to 100 |

A non-limiting example of Component 2 of the present invention comprises benzoyl peroxide at a concentration of 5 weight percent in a carboxyvinyl polymer gel.

In one aspect the invention comprises a pre-filled dual chambered delivery device wherein one chamber contains Component 1 and the second chamber contains Component 2. The dual chambered delivery device separates Component 1 and Component 2 until each Component is dispensed from the delivery device substantially at the same time to allow for admixing to form the composition of the invention. The dispensing can be achieved via a pump, either metered or non-metered. In one embodiment the delivery system comprises a top part that includes the pump assembly and actuators, and optionally an overcap; and a bottom part that includes the individual chambers enclosed in an outer casing/housing; the top part can snap on the bottom part. In a further embodiment a one-piece dispensing button works cooperatively with the actuators to allow the user to press at any spot on this button to allow the pump to dispense Component 1 and Component 2 at the same time. When the center of the button is pressed, the two gel components are dispensed at a desired volume ratio such as an approximate 1:1 ratio. This spot is optionally marked so that actuation is effected substantially consistently at the same place by end users.

A separate Component 1 of a composition of the present invention was prepared as follows.

A predetermined amount of a gelling/thickening agent was dissolved completely in a predetermined amount of purified water under agitation. This solution was combined with another solution containing predetermined amounts of tretinoin, moisturizer, humectant, antioxidant, and other optional excipients. The pH was adjusted to a range of desired values.

A separate Component 2 of a composition of the present invention was prepared as follows.

A predetermined amount of a gelling/thickening agent was dissolved completely in a predetermined amount of water under agitation. This solution was combined with another solution containing predetermined amounts of micronized penzoyl peroxide, humectant and other optional excipients. The pH was adjusted to a range of desired values.

In one embodiment, each of Components 1 and 2 is contained in a separate chamber of a two-chamber delivery system, which is a commercially available non-metered dual airless pump. The delivery system comprises a top part that includes the pump assembly, actuators, and an overcap; and a bottom part that includes the individual chambers enclosed in an outer casing/housing. The top part snaps on the bottom part.

A one-piece dispensing button works cooperatively with the actuators to allow the user to press at any spot on this button to allow the pump to dispense Component 1 and Component 2 at the same time. When the center of the button is pressed, the two gel components are dispensed at an approximate 1:1 ratio. This spot is marked so that actuation is effected substantially consistently at the same place by end users. The dispensing mechanism may be designed to dispense the two components at other ratios, if desired.

A clinical study in acne vulgaris patients was conducted to assess the safety, tolerability, and efficacy of a composition of the present invention (also referred to as “IDP-120” below) in comparison with a composition comprising only tretinoin active ingredient (at effective concentration of 0.05% tretinoin, referred to as “Component A” below), a composition comprising only benzoyl peroxide active ingredient (at effective concentration of 2.5% benzoyl peroxide, referred to as “Component B” below), and the vehicle of said composition of the present invention at weeks 2, 4, 8, and 12. Component A is an admixture of approximately equal weights of Component A₁ and Component A₂, as shown in Table 4. Component B is an admixture of approximately equal weights of Component B₁ and Component B₂, as shown in Table 5. IDP-120 is an admixture of approximately equal weights of Component A₁ and Component B₁.

TABLE 4
Component A Composition for Clinical Study

| | Concentration | |
|---|---|---|
| | (weight percent) | |
| Ingredient | Component A₁ | Component A₂ |
| Tretinoin | 0.1 | 0 |
| Glycerin | 9.63 | 0 |
| Carbopol 980 | 0.9 | 1.75 |
| Octoxynol-9 | 0.12 | 0 |
| sodium hyaluronate | 0.011 | 0 |
| soluble collagen | 8 | 0 |
| BHT | 0.021 | 0 |
| Methylparaben | 0.2 | 0.17 |
| benzyl alcohol | 0.5 | 0 |
| Propylparaben | 0 | 0.03 |
| propylene glycol | | 7.5 |
| titanium dioxide | | 0.25 |
| pH adjuster | q.s. to adjust pH to | q.s. to adjust pH to |
| | 5.4-5.5 | 5.4-5.5 |
| purified water | q.s. to 100 | q.s. to 100 |

TABLE 5
Component B Composition for Clinical Study

| | Concentration | |
|---|---|---|
| | (weight percent) | |
| Ingredient | Component B₁ | Component B₂ |
| benzoyl peroxide | 5 | 0 |
| propylene glycol | 7.5 | 0 |
| Carbopol 980 | 1.75 | 0.9 |
| Methylparaben | 0 | 0.2 |
| benzyl alcohol | 0 | 0.5 |
| Propylparaben | 0 | 0.03 |
| Glycerin | 0 | 9.63 |
| Octoxynol-9 | 0 | 0.9 |
| sodium hyaluronate | 0 | 0.11 |
| soluble collagen | 0 | 8 |
| BHT | 0 | 0.021 |
| FD&C yellow No.5 | 0 | 0.015 |
| pH adjuster | q.s. to adjust pH | q.s. to adjust pH |
| | to 5.4-5.5 | to 5.4-5.5 |
| purified water | q.s. to 100 | q.s. to 100 |

This was a multi-center, randomized, double-blind, vehicle-controlled, 12-week study. To be eligible for the study subjects must be at least 9 years of age and have a clinical diagnosis of moderate to severe acne (defined as an Evaluator's Global Severity Score (“EGSS”) of 3 or 4), presenting with 20-40 inflammatory facial lesions (papules, pustules, and nodules), 20-100 non-inflammatory facial lesions (open and closed comedones), and <2 facial nodules.

TABLE 6
Evaluator's Global Severity Score

| Score | Grade | Description |
|---|---|---|
| 0 | Clear | Normal, clear skin with no |
| | | evidence of acne vulgaris |
| 1 | Almost | Rare non-inflammatory lesions |
| | Clear | present, with rare non-inflamed |
| | | papules (papules must be resolving |
| | | and may be hyperpigmented, |
| | | though not pink-red) |
| 2 | Mild | Some non-inflammatory lesions |
| | | are present, with few inflammatory |
| | | lesions (papules/pustules only; no |
| | | nodulocystic lesions) |
| 3 | Moderate | Non-inflammatory lesions |
| | | predominate, with multiple |
| | | inflammatory lesions evident: |
| | | several to many comedones and |
| | | papules/pustules, and there may or |
| | | may not be one |
| | | nodulocystic lesion |
| 4 | Severe | Inflammatory lesions are more |
| | | apparent, many comedones and |
| | | papules/pustules, there may or |
| | | may not be up to 2 nodulocystic lesions |

The subjects were randomized in a 2:2:2:1 ratio to the following treatment groups:
Group 1: 109 Subjects to IDP-120 Gel (tretinoin and benzoyl peroxide BPO gel, 0.05%/2.5%);
Group 2: 98 Subjects to IDP-120 Component A (tretinoin gel, 0.05%);
Group 3: 108 Subjects to IDP-120 Component B (benzoyl peroxide, 2.5%); and
Group 4: 49 Subjects to IDP-120 vehicle gel.

The Group-1 subjects receive pumps that contain Component A₁ in the first chamber and Component B₁ in the second chamber. The Group-2 subjects received pumps that contain Component A₁ in the first chamber and Component A₂ in the second chamber. The Group-3 subjects receive pumps that contain Component B₁ in the second chamber and Component B₂ in the first chamber. The Group-4 subjects receive pumps that contain Component B₂ in the first chamber and Component A₂ vehicle in the second chamber.

All subjects received once daily, topically-applied treatment to the face for 12 weeks. Subject visits include Screening, Baseline, Week 2, Week 4, Week 8, and Week 12, at which safety and efficacy assessments were conducted. Subjects were evaluated for drug usage compliance at each post-baseline study visit (Weeks 2, 4, 8, and 12). Subjects applied their treatments at home, once daily, as instructed by the study coordinator or designee at each investigational center.

The investigator assessed the subject's face at each study visit. Information on reported and observed adverse events (“AEs”) was obtained at each visit.

Efficacy analyses were conducted on the Intent-to-Treat (“ITT”) (primary) and Per-Protocol (“PP”) (supportive) populations.

Primary Efficacy

The primary efficacy endpoints are intended to compare once daily application of IDP-120 Gel with IDP-120 Vehicle Gel and each of the individual gel components. Specifically, the primary efficacy endpoints include:
Absolute change in inflammatory lesion count from baseline to Week 12, as summarized using descriptive and inferential statistics;
Absolute change in non-inflammatory lesion count from baseline to Week 12, as summarized using descriptive and inferential statistics;
Proportion of subjects who have a least a 2 grade reduction at Week 12 from baseline in the Evaluator's Global Severity Score and were Clear or Almost Clear, as summarized using descriptive and inferential statistics.

Assessment of Safety

Safety was evaluated by tabulations of adverse events (“AEs”), Cutaneous Safety Evaluation, and Tolerability Evaluations. Cutaneous Safety Evaluation scores (scaling and erythema) and Tolerability (itching, burning, and stinging) are presented with descriptive statistics at Baseline and at Weeks 2, 4, 8, and 12 for each treatment group.

Scaling:

0—None: No scaling

1—Mild: Barely perceptible, fine scales present on limited areas of the face

2—Moderate: Fine scale generalized to all areas of the face

3—Severe: Scaling and peeling of skin over all areas of the face

Erythema:

0—None: No evidence of erythema present

1—Mild: Slight pink coloration

2—Moderate: Definite redness

3—Severe: Marked erythema, bright red to dusky dark red in color

Itching:

0—None: No itching

1—Mild: Slight itching, not really bothersome

2—Moderate: Definite itching that is somewhat bothersome

3—Severe: Intense itching that may interrupt daily activities and/or sleep

Burning:

0—None: No burning

1—Mild: Slight burning sensation; not really bothersome

2—Moderate: Definite warm, burning sensation that is somewhat bothersome

3—Severe: Hot burning sensation that causes definite discomfort and may interrupt daily activities and/or sleep

Stinging:

0—None: No stinging

1—Mild: Slight stinging sensation, not really bothersome

2—Moderate: Definite stinging sensation that is somewhat bothersome

3—Severe: Stinging sensation that causes definite discomfort and may interrupt daily activities and/or sleep

Clinical Efficacy was determined based on the percentage of subjects who achieved treatment successes at Week-12 visit.

The IDP-120 group was compared to each of the other treatment groups: (1) Component-A group, (2) Component-B group, and (3) Vehicle group.

To be judged as a treatment success, subjects had to show two-grade improvement in EGSS from the baseline, and to have an EGSS score of “clear,” or “almost clear” at the evaluation time. Subjects not achieving treatment success by this standard were considered treatment failures, even though such subjects may have experienced some degree of improvement in their acne vulgaris.

Results

The efficacy of IDP-120 comprising tretinoin 0.05 weight percent and benzoyl peroxide 2.5 weight percent is now shown and discussed.

Subject baseline characteristics for per-protocol population are shown in Table 7. Inflammatory and Non-Inflammatory lesion counts at Week 12 are shown in Tables 8 and 9. Evaluator's Global Severity Scores at Week 12 are shown in Table 10.

TABLE 7
Analysis of Subject Baseline Characteristics
(Per-Protocol Population)

| | | IDP-120 | IDP-120 | | |
|---|---|---|---|---|---|
| | | Com- | Com- | IDP-120 | |
| | IDP-120 | ponent | ponent | Vehicle | |
| | Gel | A | B | Gel | P- |
| | (N = 90) | (N = 72) | (N = 87) | (N = 40) | value |
| Inflammatory | | | | | |
| Lesion Count | | | | | |
| N | 90 | 72 | 87 | 40 | 0.035^{a} |
| Mean | 26.3 | 25.6 | 27.3 | 24.5 | |
| SD | 5.76 | 4.46 | 6.42 | 4.16 | |
| Median | 25.0 | 24.0 | 25.0 | 23.5 | |
| Min. to Max. | 20 to 40 | 20 to 40 | 20 to 40 | 20 to 38 | |
| Non- | | | | | |
| Inflammatory | | | | | |
| Lesion Count | | | | | |
| N | 90 | 72 | 87 | 40 | 0.584^{a} |
| Mean | 40.3 | 37.8 | 38.7 | 35.8 | |
| SD | 19.26 | 15.78 | 20.25 | 13.93 | |
| Median | 35.0 | 35.0 | 30.0 | 33.0 | |
| Min. to Max. | 20 to 99 | 20 to 88 | 20 to 100 | 20 to 83 | |
| Evaluator's | | | | | |
| Global | | | | | |
| Severity Score | | | | | |
| N | 90 | 72 | 87 | 40 | 0.333^{b} |
| 0-Clear | 0 (0.0%) | 0 (0.0%) | 0 (0.0%) | 0 (0.0%) | |
| 1-Almost Clear | 0 (0.0%) | 0 (0.0%) | 0 (0.0%) | 0 (0.0%) | |
| 2-Mild | 0 (0.0%) | 0 (0.0%) | 0 (0.0%) | 0 (0.0%) | |
| 3-Moderate | 79 (87.8%) | 66 (91.7%) | 80 (92.0%) | 39 (97.5%) | |
| 4-Severe | 11 (12.2%) | 6 (8.3%) | 7 (8.0%) | 1 (2.5%) | |

^{a}P-value from a one-way analysis of variance with factor of treatment group.
^{b}P-value from a Cochran-Mantel-Haenszel general association test.

TABLE 8
Summary of Inflammatory Lesion Counts at Each Evaluation
(Per-Protocol Population)

| | | IDP-120 | IDP-120 | IDP-120 |
|---|---|---|---|---|
| | IDP-120 | Component | Component | Vehicle |
| | Gel | A | B | Gel |
| | (N = 90) | (N = 72) | (N = 87) | (N = 40) |
| Inflammatory | | | | |
| Lesion Count | | | | |
| Week 12 | | | | |
| N | 90 | 72 | 87 | 40 |
| Mean | 12.1 | 14.0 | 14.0 | 14.8 |
| SD | 11.20 | 10.90 | 10.49 | 9.66 |
| Median | 8.0 | 11.5 | 12.0 | 12.0 |
| Min. to Max. | 0 to 49 | 0 to 48 | 0 to 58 | 0 to 37 |
| Absolute Change | | | | |
| from Baseline | | | | |
| N | 90 | 72 | 87 | 40 |
| Mean | −14.2 | −11.6 | −13.4 | −9.7 |
| SD | 10.28 | 10.03 | 9.29 | 10.08 |
| Median | −16.5 | −14.0 | −14.0 | −9.5 |
| Min. to Max. | −31 to 17 | −29 to 23 | −32 to 20 | −27 to 14 |
| Percent Change | | | | |
| from Baseline | | | | |
| N | 90 | 72 | 87 | 40 |
| Mean | −55.29 | −46.55 | −50.20 | −38.76 |
| SD | 38.547 | 41.262 | 33.415 | 40.406 |
| Median | −68.78 | −59.10 | −52.38 | −41.43 |
| Min. to Max. | −100.0 to | −100.0 to | −100.0 to | −100.0 to |
| | 68.0 | 104.5 | 52.6 | 63.6 |

Note:
Last observation carried forward used to impute missing values.

TABLE 9
Summary of Non-Inflammatory Lesion Counts at Each Evaluation
(Per-Protocol Population)

| | | IDP-120 | IDP-120 | IDP-120 |
|---|---|---|---|---|
| | IDP-120 | Component | Component | Vehicle |
| | Gel | A | B | Gel |
| | (N = 90) | (N = 72) | (N = 87) | (N = 40) |
| Non-Inflammatory | | | | |
| Lesion Count | | | | |
| Week 12 | | | | |
| N | 90 | 72 | 87 | 40 |
| Mean | 19.7 | 21.4 | 28.4 | 28.6 |
| SD | 16.12 | 17.05 | 26.51 | 22.42 |
| Median | 15.0 | 18.0 | 22.0 | 22.0 |
| Min. to Max. | 0 to 90 | 1 to 91 | 0 to 111 | 1 to 97 |
| Absolute Change | | | | |
| from Baseline | | | | |
| N | 90 | 72 | 87 | 40 |
| Mean | −20.6 | −16.3 | −10.3 | −7.2 |
| SD | 15.27 | 14.94 | 19.90 | 16.98 |
| Median | −20.0 | −16.0 | −13.0 | −8.5 |
| Min. to Max. | −68 to 9 | −79 to 23 | −89 to 41 | −34 to 55 |
| Percent Change | | | | |
| from Baseline | | | | |
| N | 90 | 72 | 87 | 40 |
| Mean | −51.62 | −44.12 | −30.15 | −23.57 |
| SD | 29.969 | 31.315 | 49.317 | 46.410 |
| Median | −55.53 | −47.30 | −45.45 | −20.24 |
| Min. to Max. | −100.0 to | −98.8 to | −100.0 to | −95.7 to |
| | 24.3 | 52.3 | 145.0 | 148.6 |

Note:
Last observation carried forward used to impute missing values.

TABLE 10
Summary of Evaluator's Global Severity Score at Each Evaluation
(Per-Protocol Population)

| | | IDP-120 | IDP-120 | IDP-120 |
|---|---|---|---|---|
| | IDP-120 | Component | Component | Vehicle |
| | Gel | A | B | Gel |
| | (N = 90) | (N = 72) | (N = 87) | (N = 40) |
| Evaluator's Global | | | | |
| Severity Score | | | | |
| Week 12 | | | | |
| N | 90 | 72 | 87 | 40 |
| 0-Clear | 5 (5.6%) | 1 (1.4%) | 3 (3.4%) | 0 (0.0%) |
| 1-Almost Clear | 20 (22.2%) | 10 (13.9%) | 16 (18.4%) | 6 (15.0%) |
| 2-Mild | 41 (45.6%) | 35 (48.6%) | 33 (37.9%) | 16 (40.0%) |
| 3-Moderate | 19 (21.1%) | 25 (34.7%) | 32 (36.8%) | 18 (45.0%) |
| 4-Severe | 5 (5.6%) | 1 (1.4%) | 3 (3.4%) | 0 (0.0%) |
| At Least Two Grade | | | | |
| Reduction | | | | |
| from Baseline and | | | | |
| Achieving Clear | | | | |
| or Almost Clear | | | | |
| N | 90 | 72 | 87 | 40 |
| Success | 25 (27.8%) | 11 (15.3%) | 19 (21.8%) | 6 (15.0%) |
| Failure | 65 (72.2%) | 61 (84.7%) | 68 (78.2%) | 34 (85.0%) |

Note:
Last observation carried forward used to impute missing values.

Treatment success was defined as at least a two-grade improvement from Baseline in the EGSS score and an EGSS score equating to “Clear” or “Almost Clear” at Week 12, and is shown in Table 11.

TABLE 11
Percentage of Subjects Achieving Treatment Success at Week 12
(Per-Protocol Population)

| IDP-120 | Component A | Component B | Vehicle |
|---|---|---|---|
| 27.8% | 15.3% | 21.8% | 15.0% |

Actual Clinical Efficacy compared to Predicted Additive Clinical Efficacy (as a percentage of subjects achieving “Treatment Success”) is shown in Table 12. The “Treatment Success” percentages for the active treatment groups (IDP-120, Component A, and Component B) were corrected for vehicle effect by subtracting the actual Vehicle group results from each to determine the net Treatment Success are shown as a percentage of the number of subjects treated.

TABLE 12
Comparative Treatment Success Rates for IDP-120, Component A,
and Component B (Corrected for Vehicle Effect)

| IDP-120 | Component A | Component B |
|---|---|---|
| 12.8% | 0.3% | 6.8% |

The synergistic effect of IDP-120 is illustrated by comparing the clinical efficacy from IDP-120 to the predicted efficacy from combining Component A and Component B. The vehicle-adjusted percentage of patients who were successfully treated with IDP-120 was 12.8%, which is greater than the sum of vehicle-adjusted percentages of patients who were successfully treated singly with Component A and Component B (0.3%+6.8% or 7.1%).

The mean scores of itching, burning, and stinging, as reported by the subjects at Week-12 evaluation are shown in Table 13.

TABLE 13
Mean Scores of Itching, Burning, and Stinging for IDP-120,
Component A, Component B, and Vehicle

| Adverse | | Component | Component | |
|---|---|---|---|---|
| Event | IDP-120 | A | B | Vehicle |
| Itching | 0.06 | 0.16 | 0.09 | 0.05 |
| Burning | 0.18 | 0.11 | 0.05 | 0.00 |
| Stinging | 0.12 | 0.05 | 0.03 | 0.00 |

The mean scores of itching, burning, and stinging for IDP-120, Component A, and Component B, corrected for the effect of vehicle are shown in Table 14.

TABLE 14
Mean Scores of Itching, Burning, and Stinging for IDP-120,
Component A, and Component B, Corrected for the Effect of Vehicle

| Adverse | | | | Component A + |
|---|---|---|---|---|
| Event | IDP-120 | Component A | Component B | Component B |
| Itching | 0.01 | 0.11 | 0.04 | 0.15 |
| Burning | 0.18 | 0.11 | 0.05 | 0.16 |
| Stinging | 0.12 | 0.05 | 0.03 | 0.08 |

The mean scores of scaling, erythema, hypopigmentation, and hyperpigmentation, at Week-12 evaluation for IDP-120, Component-A, Component-B, and Vehicle groups are shown in Table 15.

TABLE 15
Mean Scores of Scaling, Erythrema, Hypopigmentation,
and Hyperpigmentation

| Adverse Reaction | IDP-120 | Component A | Component B | Vehicle |
|---|---|---|---|---|
| Scaling | 0.15 | 0.13 | 0.10 | 0.07 |
| Erythema | 0.23 | 0.19 | 0.15 | 0.16 |
| Hypopigmentation | 0.02 | 0.06 | 0.03 | 0.00 |
| Hyperpigmentation | 0.17 | 0.17 | 0.19 | 0.16 |

The mean scores of scaling, erythrema, hypopgmentation, and hyperpigmentation for IDP-120, Component A, and Component B corrected for the effect of the vehicle are shown in Table 16.

TABLE 16
Mean Scores of Scaling, Erythrema, Hypopigmentation, and
Hyperpigmentation, Corrected for the Effect of the Vehicle

| | | Com- | Com- | Component |
|---|---|---|---|---|
| Adverse | | ponent | ponent | A + |
| Reaction | IDP-120 | A | B | Component B |
| Scaling | 0.08 | 0.06 | 0.03 | 0.09 |
| Erythema | 0.07 | 0.03 | −0.01^{(a)} | 0.03 |
| Hypopigmentation | 0.02 | 0.06 | 0.03 | 0.09 |
| Hyperpigmentation | 0.01 | 0.01 | 0.03 | 0.04 |

Notes:
^{(a)}negative values are taken to be zero for the purposes of comparison.

Surprisingly, the corrected mean scores for itching, scaling, hypopigmentation, and hyperpigmentation for IDP-120 are lower than those predicted for the combination of Component A and Component B. Thus, IDP-120 is synergistically beneficial with respect to these adverse reactions.

In another aspect, the present invention provides a method for treating acne vulgaris. The method comprises topically applying to an affected area of the body of a subject suffering from acne vulgaris any one of the compositions of the present invention, as disclosed herein, one or more times per day for a period of time sufficient to treat such acne vulgaris. For example, such a period of time may be 1 to 12 weeks, or 1 to 24 weeks, or longer as needed. For example, such a period of time may be one week, two weeks, four weeks, eight weeks, twelve weeks, eighteen weeks, twenty-four weeks, or longer as needed. For example, a composition of the present invention is applied topically to affected areas of the body once per day for 12 weeks. Alternatively, it may be applied two or three times per day for 1-12 weeks. Alternatively, it may be applied once per day for one week to six months. For example, it may be applied once per day for two weeks, four weeks, eight weeks, twelve weeks, eighteen weeks, or twenty-four weeks.

In yet another aspect, the present invention provides a method of treating acne vulgaris topically with pharmaceutical composition comprising a combination of: (a) tretinoin at a concentration of about 0.01-0.1 weight percent of the composition; and (b) benzoyl peroxide at a concentration of about 1-10 weight percent of the composition; wherein the composition is administered topically to an affected area of a subject in an amount, at a frequency, and for a period of time sufficient to treat said acne vulgaris; and wherein the combination provides synergistic clinical efficacy, as measured by treatment success.

In yet another aspect, the present invention provides a method of treating acne vulgaris topically with pharmaceutical composition comprising a combination of: (a) tretinoin at a concentration of about 0.01-0.1 weight percent of the composition; and (b) benzoyl peroxide at a concentration of about 1-10 weight percent of the composition; wherein the composition is administered topically to an affected area of a subject in an amount, at a frequency, and for a period of time sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.03-0.08 (or 0.04-0.07, or 0.01-0.06) weight percent of the composition; and (b) benzoyl peroxide at a concentration of about 1-5 (or 1.5-4, or 2-3) weight percent of the composition; wherein the composition is administered topically to an affected area of a subject in an amount, at a frequency, and for a period of time sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.03-0.08 (or 0.04-0.07, or 0.01-0.06) weight percent of the composition; and (b) benzoyl peroxide at a concentration of about 1-5 (or 1.5-4, or 2-3) weight percent of the composition; wherein the composition is administered topically to an affected area of a subject one or more times per day for 1-24 weeks, in an amount sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.05 weight percent of the composition; and (b) benzoyl peroxide at a concentration of about 2.5 weight percent of the composition; wherein the composition is administered topically to an affected area of a subject 1-4 times per day for 1-24 weeks, in an amount sufficient to treat said acne vulgaris sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.05 weight percent of the composition; and (b) benzoyl peroxide at a concentration of about 2.5 weight percent of the composition; wherein the composition is administered topically to an affected area of a subject one or two times per day for 2, 4, 6, 8, 10, or 12 weeks, in an amount sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.05 weight percent of the composition; and (b) benzoyl peroxide at a concentration of about 2.5 weight percent of the composition; wherein the composition is administered topically to an affected area of a subject once per day for 12 weeks, in an amount sufficient to treat said acne vulgaris; and wherein the clinical success rate of the combination is synergistic compared to the clinical success rate of the tretinoin component at the same concentration used alone plus the clinical success rate of the benzoyl peroxide component used alone at the same concentration.

In yet another aspect, the present invention provides a method of treating acne vulgaris topically with pharmaceutical composition comprising a combination of: (a) tretinoin at a concentration of about 0.05 weight percent of the composition; and (b) benzoyl peroxide at a concentration of about 2.5 weight percent of the composition; wherein the composition is administered topically to an affected area of a subject once per day for 12 weeks, in an amount sufficient to treat said acne vulgaris; wherein the clinical success rate of the combination is synergistic compared to the clinical success rate of the tretinoin component at the same concentration used alone plus the clinical success rate of the benzoyl peroxide component used alone at the same concentration; and wherein the method provides a synergistically beneficial effect with respect to at least one adverse reaction. In one embodiment, such adverse reaction is selected from the group consisting of itching, scaling, hypopigmentation, and hyperpigmentation.

It is also contemplated that in certain circumstances, a period of non-treatment may be allowed between two periods of treatment with a composition of the present invention.

In still another aspect, such amount sufficient to treat acne vulgaris is about 0.5-2 gram per application. In one embodiment, such amount sufficient to treat acne vulgaris is about 0.7 gram per application.

In still another aspect, a composition of the present invention may be used in conjunction with another method of treatment of acne vulgaris, such as a topical antibiotic drug (e.g., clindamycin), an oral antibiotic drug, or a topical or oral anti-inflammatory drug.

While the present disclosure shows and describes a number of exemplary embodiments, it will be manifest to those skilled in the art that various further modifications may be made without departing from the spirit and scope of the underlying inventive concept and that the same is not limited to particular compositions, processes, methods, or structures herein shown and described.

What is claimed is:

1. A method of treating acne vulgaris, comprising: administering topically a therapeutically effective amount of a composition comprising tretinoin at a concentration of about 0.01-0.1 weight percent and benzoyl peroxide at a concentration of about 1-5 weight percent, to an affected area, at a frequency and for a period of time sufficient to treat said acne vulgaris; wherein said concentrations are based on weight percent of the composition, and said tretinoin and benzoyl peroxide are present together in the composition.

2. The method of claim 1; wherein the tretinoin concentration is about 0.04-0.06 weight percent, and the benzoyl peroxide concentration is about 2-3 weight percent.

3. The method of claim 1; wherein the tretinoin concentration is about 0.05 weight percent, and the benzoyl peroxide concentration is about 2.5 weight percent.

4. The method of claim 1; wherein said administering is carried out 1 or 2 times daily for 1-24 weeks.

5. The method of claim 3; wherein said administering is carried out once daily for 12 weeks.

6. The method of claim 3; wherein said administering is carried out once daily for 24 weeks.

7. The method of claim 3; wherein said composition is an admixture of a first composition comprising tretinoin and a second composition comprising benzoyl peroxide.

8. The method of claim 7; wherein said first composition and said second composition are admixed substantially immediately prior to said administering.

9. A topical pharmaceutical composition for treating acne vulgaris, comprising: tretinoin at a concentration of about 0.01-0.1 weight percent of the composition and benzoyl peroxide at a concentration of about 1-5 weight percent of the composition.

10. The composition of claim 9; wherein the tretinoin concentration is about 0.04-0.06 weight percent of the composition, and the benzoyl peroxide concentration is about 2-3 weight percent of the composition.

11. The composition of claim 9; wherein the tretinoin concentration is about 0.05 weight percent of the composition, and the benzoyl peroxide concentration is about 2.5 weight percent of the composition

12. The composition of claim 9; wherein the composition is a gel.

13. The composition of claim 12; wherein said gel is an aqueous gel.

14. The composition of claim 9; wherein said composition comprises an admixture of a first amount of a first composition comprising tretinoin and a second mount of a second composition comprising benzoyl peroxide; wherein a tretinoin concentration of said first composition and a benzoyl peroxide concentration of said second composition are sufficient to provide tretinoin at about 0.05 weight percent and benzoyl peroxide at about 2.5 weight percent of said admixture.

15. The composition of claim 14; wherein weights of said first amount and said second amount are approximately equal.

16. The composition of claim 9; wherein said composition comprises an admixture of a first composition comprising tretinoin at a concentration of about 0.1 weight percent, and a second composition comprising benzoyl peroxide at a concentration of about 5 weight percent.

17. The composition of claim 16; wherein the tretinoin of the composition is stable at 32 degrees C., for about 24 hours.

18. The composition of claim 16; wherein the tretinoin of the composition is stable at 32 degrees C., for about 24 hours under room light condition.

19. The composition of claim 16; wherein the tretinoin of the composition is stable at room condition for about 24 hours.

20. The composition of claim 16; wherein more than 95 percent of the tretinoin of the composition remains after 24 hours at 32 degrees C. under room light.
